(19) United States
(12) Patent Application Publication
(10) Pub. No.: US 2020/0100937 A1
(43) Pub. Date: Apr. 2, 2020

(54) NECK COOLING NECKLACE

(71) Applicant: Barbie Patterson, Charlotte, NC (US)

(72) Inventor: Barbie Patterson, Charlotte, NC (US)

(21) Appl. No.: 16/148,172

(22) Filed: Oct. 1, 2018

(51) Int. Cl. A61F 7/10; A44C 15/00

(52) U.S. Cl. CPC A61F 7/10; A61F 2007/0075; A44C 15/005

(57) ABSTRACT

A necklace to provide a cooling effect for a user, the necklace including a necklace main body to wrap around a neck of the user, a clasp disposed at a first end of the necklace main body, and a cooling device disposed at a second end of the necklace main body to connect to the clasp, the cooling device including a cooling device main body to contact a back of the neck of the user at back portion thereof, a sensor disposed on at least one of the cooling device main body and the necklace main body to sense a temperature of the neck of the user, a microprocessor to receive a signal from the sensor in response to the sensor sensing that the temperature of the back of the neck of the user has exceeded 98.6 degrees Fahrenheit, and a thermo-electric cold plate disposed at the back portion of the cooling device main body to receive another signal from the microprocessor in response to the signal received by the microprocessor from the sensor, and to decrease in temperature such that the back of the neck of the user receives the cooling effect from the back portion of the cooling device main body.

### BACKGROUND

### 1. Field

The present general inventive concept relates generally to a necklace, and particularly, to a neck cooling necklace.

### 2. Description of the Related Art

During and after menopause many women often experience hormonal changes that lead to changes in body temperature like hot flashes. Currently, on the market, there is medication to treat hormonal changes, but there is a lack of devices that aid treating hot flashes without medication. Furthermore, the devices on the market that provide cooling effects for women with hot flashes are bulky, and cannot be worn at work or at social events.

Therefore, there is a need for a necklace that provides a cooling effect for users, while still being stylish.

### SUMMARY

The present general inventive concept provides a neck cooling necklace.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a necklace to provide a cooling effect for a user, the necklace including a necklace main body to wrap around a neck of the user, a clasp disposed at a first end of the necklace main body, and a cooling device disposed at a second end of the necklace main body to connect to the clasp, the cooling device including a cooling device main body to contact a back of the neck of the user at back portion thereof, a sensor disposed on at least one of the cooling device main body and the necklace main body to sense a temperature of the neck of the user, a microprocessor to receive a signal from the sensor in response to the sensor sensing that the temperature of the back of the neck of the user has exceeded 98.6 degrees Fahrenheit, and a thermo-electric cold plate disposed at the back portion of the cooling device main body to receive another signal from the microprocessor in response to the signal received by the microprocessor from the sensor, and to decrease in temperature such that the back of the neck of the user receives the cooling effect from the back portion of the cooling device main body.

The necklace may further include a battery to provide power to the sensor, the microprocessor, and the thermo-electric cold plate, and a USB port to allow the battery to charge when the USB port is connected to an external electrical outlet.

The thermo-electric cold plate may cool to a temperature of 70 degrees Fahrenheit until the sensor senses that the neck of the user has reached 98.6 degrees Fahrenheit.

### BRIEF DESCRIPTION OF THE DRAWINGS

These and/or other features and utilities of the present generally inventive concept will become apparent and more readily appreciated from the following description of the embodiments, taken in conjunction with the accompanying drawings of which:

FIG. 1 illustrates a rear perspective view of a necklace including a cooling device, according to an exemplary embodiment of the present general inventive concept; and

FIG. 2 illustrates an exploded top perspective view of the cooling device, according to an exemplary embodiment of the present general inventive concept.

### DETAILED DESCRIPTION

Various example embodiments (a.k.a., exemplary embodiments) will now be described more fully with reference to the accompanying drawings in which some example embodiments are illustrated. In the figures, the thicknesses of lines, layers and/or regions may be exaggerated for clarity.

Accordingly, while example embodiments are capable of various modifications and alternative forms, embodiments thereof are shown by way of example in the figures and will herein be described in detail. It should be understood, however, that there is no intent to limit example embodiments to the particular forms disclosed, but on the contrary, example embodiments are to cover all modifications, equivalents, and alternatives falling within the scope of the disclosure. Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

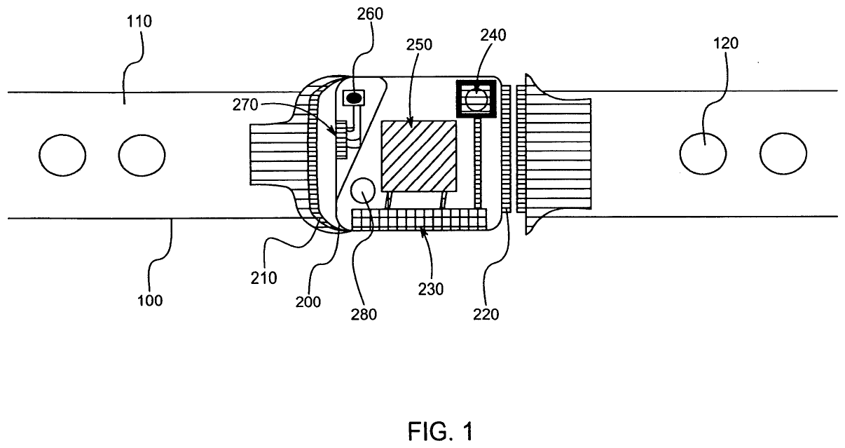

FIG. 1 illustrates a front perspective view of a necklace 100 including a cooling device 200, according to an exemplary embodiment of the present general inventive concept.

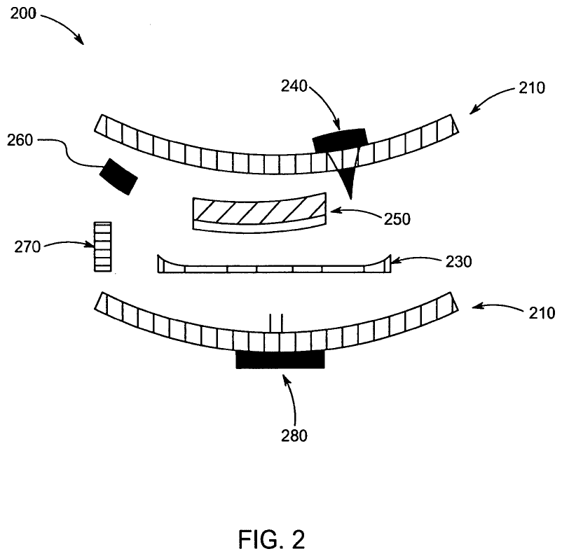

FIG. 2 illustrates an exploded top perspective view of the cooling device 200, according to an exemplary embodiment of the present general inventive concept.

Referring to FIGS. 1 and 2, the necklace 100 may include a necklace main body 110, at least one accessory 120, and a clasp 130, but is not limited thereto.

The necklace main body 110 may be constructed from any type of material known to one of ordinary skill in the art that may be part of a necklace, including, but not limited to, cloth, metal, precious metals (e.g., gold, silver, platinum, etc.), precious stones (e.g., diamonds, pearls, rubies, emeralds, topaz, onyx, etc.), beads, string, rope, cord, rubber, plastic, etc.

The necklace main body 110 may wrap around a user's neck, and may be designed as a choker necklace, a hanging necklace, a collar necklace, a thread necklace, a matinee necklace, a bib necklace, a string necklace, an opera necklace, a princess necklace, or any other type of necklace known to one of ordinary skill in the art.

The at least one accessory 120 may be disposed on a surface of the necklace main body 110, and may include any type of accessory known to one of ordinary skill in the art that may be typically found on a necklace, including, but not limited to, precious stones (e.g., diamonds, pearls, rubies, emeralds, topaz, onyx, etc.), beads, and precious metals (e.g., gold, silver, platinum, etc.).

The clasp 130 may be disposed at a first end of the necklace main body 110, and may connect to the cooling device 200, which may be disposed at a second end of the necklace main body 110.

The cooling device 200 may include a cooling device main body 210, a clasp receiving member 220, a microprocessor 230, a thermo-sensor 240, thermo-electric cold plate 250, a battery 260, a USB port 270, and a button 280, but is not limited thereto.

The cooling device main body 210 may have any shape known to one of ordinary skill in the art, and may include the clasp receiving member 220, the microprocessor 230, the thermo-sensor 240, the thermo-electric cold plate 250, the battery 260, and the USB port 270 disposed therein and/or thereon.

Furthermore, the main body 210 may be slightly concave in order to sit comfortably on a back portion of a user's neck. As such, the cooling device main body 210 may contact the back of the neck of the user at back portion thereof.

The clasp receiving member 220 may be disposed at a side portion of the cooling device main body 210 to correspond to the clasp 130 of the necklace 100. As such, when the necklace 100 is placed around a user's neck and the clasp 130 is connected to the clasp receiving member 220, the cooling device 200 may be disposed at a back portion of the user's neck.

The clasp 130 and the clasp receiving member 220 may be a clasp/clasp receiving member combination of any type known to one of ordinary skill in the art. Specifically, the clasp 130 and the clasp receiving member 220 may be a pin and tuck connection closure at a first portion, and a spring-lock connector at a second portion.

The microprocessor 230 may be any type of central processing unit, processor, controller, computer, or any type of computing unit that may receive signals from components within the cooling unit 200, and/or emit signals to components within the cooling unit 200.

The thermo-sensor 240 may be disposed on the cooling device main body 210 (e.g., a back portion of the cooling device main body 210), or alternatively, may be disposed on the necklace main body 110.

The thermo-sensor 240 may be a heat sensor that may sense a temperature of the user's body (i.e., the user's neck), when the user is wearing the necklace 100. If the temperature of the user's body exceeds 98.6 degrees, for example, the thermo-sensor 240 may send a signal to the microprocessor 230, which may then in response to the signal received from the thermo-sensor 240, send a signal to the thermo-electric cold plate 250 to cause the thermo-electric cold plate 250 to begin cooling. When the thermo-electric cold plate 250 becomes cooler, and the user is wearing the necklace 100, the user may receive a cooling sensation on the back of the user's neck. In other words, the thermo-electric cold plate 250 may be disposed inside a back portion of the cooling device main body 210, or alternatively, may be the back portion of the cooling device main body 210 such that the thermo-electric cold plate 250 may directly contact the back of the neck of the user.

There may be a cooling cycle programmed into the microprocessor 230 to control the cooling of the thermo-electric cold plate 250. As such, the cooling cycle may be as follows: When the thermo-electric cold plate 250 is activated and begins cooling, an induction cycle of a cooling period may be 1 minute. Within this time frame the cooling of the thermo-electric cold plate 250 may drop to 70 degrees Fahrenheit, thereby providing a gentle cooling sensation to a back portion of the user's neck. As such, a constant cool temperature will remain at 70 degrees Fahrenheit until the body's temperature is at 98.6 F.

Therefore, the thermo-sensor 240 may sense a temperature of the back of the neck of the user, and then send a first signal to the microprocessor 230 when the thermos-sensor 240 has sensed that the temperature of the back of the neck of the user has exceeded 98.6 degrees Fahrenheit.

When the microprocessor 230 receives the first signal from the thermo-sensor 240 in response to the thermo-sensor 240 sensing that the temperature of the back of the neck of the user has exceeded 98.6 degrees Fahrenheit, the microprocessor 230 may send a second signal to the thermo-electric cold plate 250.

When the thermo-electric cold plate 250 receives the second signal from the microprocessor 230 in response to the first signal received by the microprocessor 230 from the thermo-sensor 240, the thermo-electric cold plate 250 may decrease in temperature such that the back of the neck of the user receives the cooling effect from the back portion of the cooling device 200.

The battery 260 may be a rechargeable battery that provides power to the microprocessor 230, the thermo-sensor 240, and the thermo-electric cold plate 250.

The USB port 270 may be any type of USB port, including, a mini USB port, a micro USB port, a regular USB port, an IPHONE port, or any other type of port to receive a charging cord therein to allow the battery 260 to be charged when the USB port 270 is connected to an electrical outlet.

The button 280 may be disposed on a surface of the cooling device main body 210, and may provide power to the cooling device 200 by connecting the battery 260 to the microprocessor 230, the thermo-sensor 240, and the thermo-electric cold plate 250. As such, pressing the button 280 a first time may turn the cooling device 200 on, and pressing the button 280 a second time may turn the cooling device 200 off.

Additionally, the cooling device 200 may include an on/off switch/button to turn on the thermo-electric cold plate, as well as a regulating knob/bar/slide/dial to allow the user to regulate how cool the thermo-electric cold plate becomes.

Furthermore, the electrical components in the present general inventive concept may be connected with wires, or via wireless communication, such as BLUETOOTH, RFID, WIFI, or any other interconnection method.

The necklace 100 and the cooling device 200 is a customized necklace thermo-electric cooling system. Its unique size creates a system that is aesthetically sensible to those who struggle with the uncomfortable symptoms of menopause. This system can be used as an alternative approach for the discomfort of hot-flashes, or it can be used as a complementary approach to this sometimes-debilitating period in a woman's life. Also, both men and women may use this system to become cooler on a hot day.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. A necklace to provide a cooling effect for a user, the necklace comprising:
a necklace main body to wrap around a neck of the user;
a clasp disposed at a first end of the necklace main body; and
a cooling device disposed at a second end of the necklace main body to connect to the clasp, the cooling device comprising:
a cooling device main body to contact a back of the neck of the user at back portion thereof,
a sensor disposed on at least one of the cooling device main body and the necklace main body to sense a temperature of the neck of the user,
a microprocessor to receive a signal from the sensor in response to the sensor sensing that the temperature of the back of the neck of the user has exceeded 98.6 degrees Fahrenheit, and
a thermo-electric cold plate disposed at the back portion of the cooling device main body to receive another signal from the microprocessor in response to the signal received by the microprocessor from the sensor, and to decrease in temperature such that the back of the neck of the user receives the cooling effect from the back portion of the cooling device main body.

2. The necklace of claim 1, further comprising:
a battery to provide power to the sensor, the microprocessor, and the thermo-electric cold plate; and
a USB port to allow the battery to charge when the USB port is connected to an external electrical outlet.

3. The necklace of claim 1, wherein the thermo-electric cold plate cools to a temperature of 70 degrees Fahrenheit until the sensor senses that the neck of the user has reached 98.6 degrees Fahrenheit.
